(19) United States
(12) Patent Application Publication
(10) Pub. No.: US 2007/0299491 A1
(43) Pub. Date: Dec. 27, 2007

(54) DRUG-ELUTING COATING ON SHOCKING COIL OF TACHY LEAD AND METHODS RELATED THERETO

(76) Inventors: Harshad Borgaonkar, Blaine, MN (US); Michael J. Pederson, Minneapolis, MN (US)

Correspondence Address: SCHWEGMAN, LUNDBERG & WOESSNER, P.A., P.O. BOX 2938, MINNEAPOLIS, MN, 55402, US

(21) Appl. No.: 11/425,889

(22) Filed: Jun. 22, 2006

(51) Int. Cl. A61N 1/00

(52) U.S. Cl. 607/120

(57) ABSTRACT

A tachy lead includes a lead body extending from a lead proximal end portion to a lead distal end portion and having an intermediate portion therebetween, one or more tissue sensing/stimulation electrodes disposed along the lead body, one or more terminal connections disposed along the lead proximal end portion. The lead further includes one or more conductors contained within the lead body extending between the tissue sensing/stimulation electrodes and the terminal connections, a porous drug-eluting coating disposed onto at least a portion of the lead body and/or sensing/stimulation electrodes, wherein the drug-eluting coating comprises porous polytetrafluoroethylene (PTFE), a biodegradable polymer and one or more drugs.

### TECHNICAL FIELD

Embodiments relate to porous drug-eluting coatings for leads, and specifically, to porous drug-eluting coatings for defibrillation shocking coils of tachy leads.

### BACKGROUND

Leads represent the electrical link between an implantable medical device (referred to as “IMD”), such as a pacer or defibrillator, and a subject's cardiac or other bodily tissue, which is to be sensed or stimulated. A lead generally includes a lead body that contains one or more electrical conductors extending from a proximal end portion of the lead to an intermediate or distal end portion of the lead. The lead body includes insulating material for covering and electrically insulating the electrical conductors. The proximal end of the lead further includes an electrical connector assembly couplable with the IMD, while the intermediate or distal end portions of the lead include one or more electrodes that may be placed within or near a desired sensing or stimulation site within the body of the subject.

An example of an IMD may be an implantable cardioverter defibrillator (ICD), which delivers electrical energy to the heart to slow it down to a more normal rhythm. An ICD can use one or more types of energy to help a heart beat normally again. The ICD may be coupled to a tachy lead utilizing one or more defibrillation shocking coils, which deliver the electrical energy. When such electrical stimulation is delivered, adjacent tissue may be injured.

### SUMMARY

Embodiments relate to a tachy lead comprising a lead body extending from a lead proximal end portion to a lead distal end portion, and having an intermediate portion therebetween, one or more tissue sensing/stimulation electrodes disposed along the lead body, one or more terminal connections disposed along the lead proximal end portion, one or more conductors contained within the lead body extending between the tissue sensing/stimulation electrodes and the terminal connections, a porous drug-eluting coating disposed onto at least a portion of the lead body and/or sensing/stimulation electrodes, wherein the porous drug-eluting coating comprises porous polytetrafluoroethylene (PTFE), a biodegradable polymer and one or more drugs.

Embodiments further provide a lead system comprising one or more tachy leads, in which each lead comprises a lead body, one or more electrodes disposed along the lead body, a porous drug-eluting coating disposed onto at least a portion of the lead body and/or electrodes, wherein the porous drug-eluting coating comprises porous polytetrafluoroethylene (PTFE), a biodegradable polymer and one or more drugs, and an implantable medical device, electrically coupled to the one or more tachy leads.

A method to manufacture a lead may comprise forming a lead and disposing a porous drug-eluting coating on all or a portion of the lead, wherein the porous drug-eluting coating comprises porous polytetrafluoroethylene (PTFE), a biodegradable polymer and one or more drugs.

### BRIEF DESCRIPTION OF THE DRAWINGS

In the drawings, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a schematic view illustrating an implantable lead system and an environment in which the lead system may be used, according to some embodiments.

FIG. 2 is a schematic view illustrating an implantable lead system for delivering or receiving signals to or from a heart, according to some embodiments.

FIG. 3 is a plan view of an implantable lead, according to some embodiments.

FIG. 4 is a cross-sectional view of an implantable lead taken along line 4-4 of FIG. 3, according to some embodiments.

FIG. 5 is a method of manufacturing a lead, according to some embodiments.

### DETAILED DESCRIPTION

The following detailed description includes references to the accompanying drawings, which form a part of the detailed description. The drawings show, by way of illustration, specific embodiments in which the present leads and methods may be practiced. These embodiments, which are also referred to herein as “examples,” are described in enough detail to enable those skilled in the art to practice the present leads and methods. The embodiments may be combined, other embodiments may be utilized or structural and logical changes may be made without departing from the scope of the present leads and methods. It is also to be understood that the various embodiments of the present leads and methods, although different, are not necessarily mutually exclusive. For example, a particular feature, structure or characteristic described in one embodiment may be included within other embodiments. The following detailed description is, therefore, not to be taken in a limiting sense, and the scope of the present leads and methods are defined by the appended claims and their legal equivalents.

In this document the terms “a” or “an” are used to include one or more than one; the term “or” is used to refer to a nonexclusive or, unless otherwise indicated; and the term “subject” is used synonymously with the term “patient.” In addition, it is to be understood that the phraseology or terminology employed herein, and not otherwise defined, is for the purpose of description only and not of limitation.

Furthermore, all publications, patents, and patent documents referred to in this document are incorporated by reference herein in their entirety, as though individually incorporated by reference. In the event of inconsistent usages between this document and those documents so incorporated by reference, the usage in the incorporated references should be considered supplementary to that of this document; for irreconcilable inconsistencies, the usage in this document controls.

Embodiments of the present invention relate to porous drug-eluting coatings for leads, such as tachy leads. The coating may provide therapy for damaged tissue in response to electric shock. The rate and release mechanisms of the one or more drugs contained in the coating may be controlled by the selection of the biodegradable polymer and its structure. The coating may also reduce defibrillation thresholds and improve biological response to shocking.

The following text and associated figures begin with a generalized discussion of a lead system (including one or more leads and a medical device), and an environment in which the lead system may be used. Although the following discusses many lead characteristics individually or in specific combinations, any combination of the lead characteristics described herein is within the scope of the present subject matter.

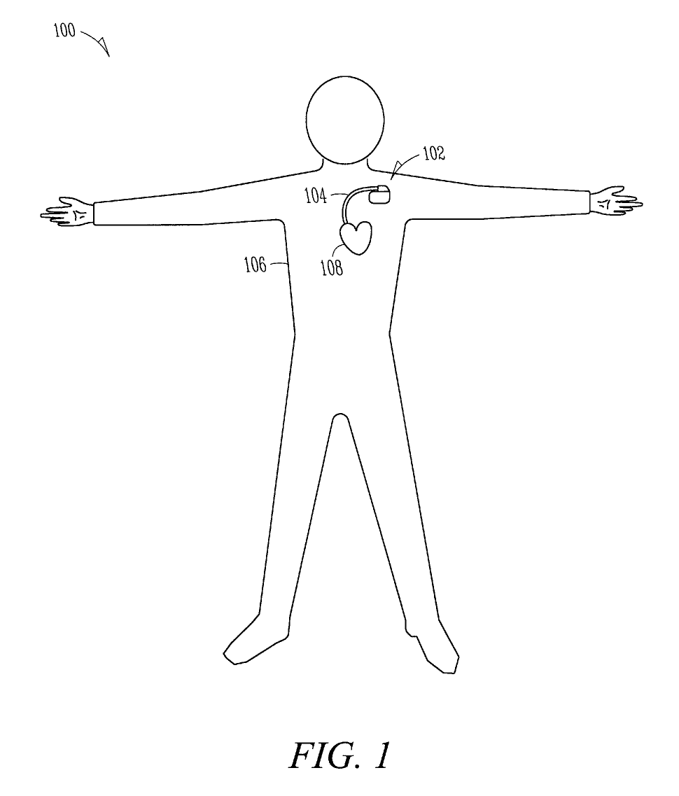

Referring to FIG. 1, a lead system 100 and a subject 106 in which lead system 100 may be used is shown, according to some embodiments of the invention. In varying examples, lead system 100 may be used for delivering or receiving electrical pulses or signals to stimulate or sense a heart 108 of a subject 106. As shown in FIG. 1, lead system 100 includes an IMD 102 and an implantable lead 104. IMD 102 generically represents, but is not limited to, cardiac function management (referred to as “CFM”) systems such as pacers, cardioverters/defibrillators, pacers/defibrillators, biventricular or other multi-site resynchronization or coordination devices such as cardiac resynchronization therapy (referred to as “CRT”) devices, sensing instruments, or drug delivery systems.

Among other things, IMD 102 includes a source of power as well as an electronic circuitry portion. In one example, the electronic circuitry includes microprocessors to provide processing, evaluation, and to determine and deliver electrical shocks or pulses of different energy levels and timing for ventricular defibrillation, cardioversion, or pacing of heart 108 in response to sensed cardiac arrhythmia including fibrillation, tachycardia, or bradycardia. In another example, IMD 102 is a battery-powered device that senses intrinsic signals of heart 108 and generates a series of timed electrical discharges.

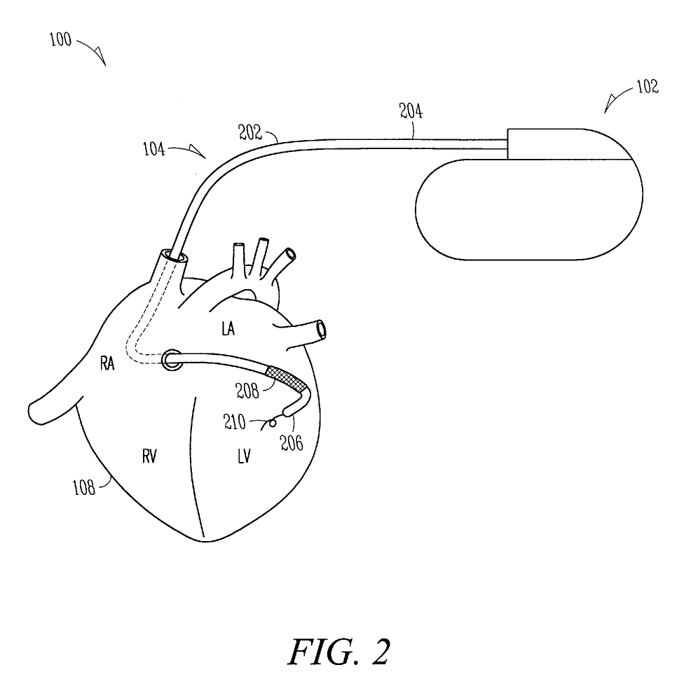

Referring to FIG. 2, a schematic view of a lead system 100 including an IMD 102 and an implantable lead 104 is shown, according to some embodiments of the invention. Lead 104 includes a lead body 202 extending from a lead proximal end portion 204, where it is couplable with IMD 102. Lead 104 extends to a lead distal end portion 206, which is positioned within, on, or near a heart 108 when implanted. As shown, lead distal end portion 206 includes at least one electrode 208, 210 that electrically links lead 104 with heart 108. Electrode 208 may be a defibrillation shocking coil electrode, for example. Electrode 210 may be a counter electrode that also assists in fixating the lead, such as an anode or cathode, for example. At least one conductor electrically couples electrodes 208 and 210 with lead proximal end portion 204 and thus, IMD 102. The conductors carry electrical current in the form of pulses or shocks between IMD 102 and electrodes 208 and 210. Lead 104 may be installed using either over-the-wire (referred to as “OTW”) or non-OTW techniques, such as stylet driving or catheter delivering.

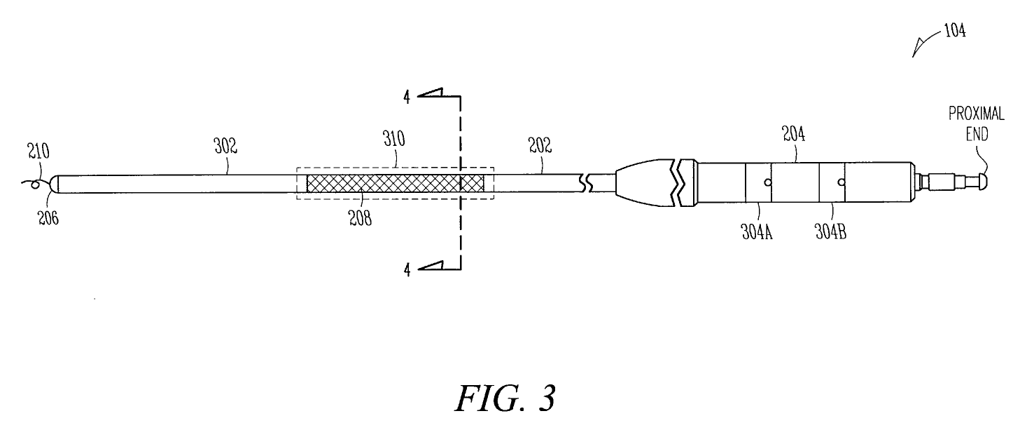

Referring to FIG. 3, a plan view of an implantable lead 104 is shown, according to some embodiments of the invention. As shown, lead 104 includes a lead body 202 extending from a lead proximal end portion 204 to a lead distal end portion 206 and having an intermediate portion 302 therebetween. In one example, lead body 202 comprises biocompatible tubing such as medical grade polyurethane. In another example, lead body 202 comprises medical grade silicone rubber. As discussed above in association with FIG. 1, a lead system 100 includes, among other things, lead 104 for electrically coupling an IMD 102 (FIG. 1) to bodily tissue, such as a heart 108 (FIG. 1), which is to be sensed or stimulated by one or more electrodes 208 and 210. It should also be understood that the lead 104 may also include means for sensing other physiological parameters, such as pressure, oxygen saturation, temperature, or the like.

As shown in FIG. 3, lead proximal end portion 204 includes one or more terminal connections 304 disposed therealong. Electrodes 208 and 210 may each be adapted to sense or stimulate heart 108 (FIG. 1) and are electrically coupled to terminal connections 304A and 304B via one or more conductors contained within lead body 202, such as in one or more longitudinally extending lumens. Lead proximal end portion 204 and terminal connections 304A and 304B disposed therealong are sized and shaped to couple to a multi-pole connector cavity, which may be incorporated into a header of IMD 102. It is through the coupling between lead proximal end portion 204 and the multi-pole connector cavity that electrodes 208 and 210 are electrically coupled to electronic circuitry of IMD 102.

A porous drug-eluting coating 310 may cover all or portions of the lead 104, including the one or more electrodes 208 and 210, the lead body 202, lead proximal end portion 204, lead distal end portion 206, or intermediate portion 302, for example. As an example, electrode 208 may be shown as a defibrillation shocking coil electrode, in which the porous drug-eluting coating 310 may partially or fully surround.

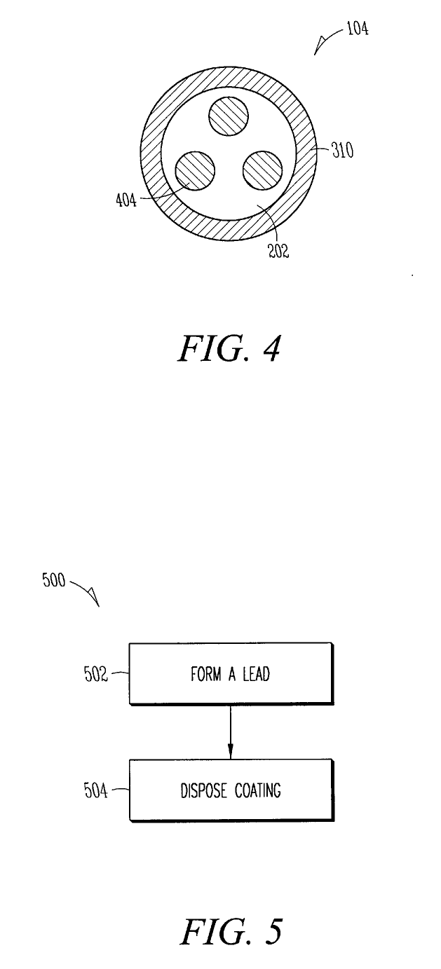

Referring FIG. 4, an exemplary cross-sectional configuration of a lead body 202 is shown, according to some embodiments of the invention. The lead body 202 may comprise one or more lumens 404 and may be surrounded by a porous drug-eluting matrix coating 310.

Referring to FIG. 5, a method 500 of manufacturing a lead is shown, according to some embodiments of the invention. A lead may be formed 502. A porous drug-eluting coating 310 may be disposed 504 on all or a portion of the lead 104.

The porous drug-eluting coating 310 may be disposed by techniques such as spraying, dipping, sputtering and/or brushing, or combinations thereof. The porous drug-eluting coating 310 may comprise porous polytetrafluoroethylene (PTFE) as the structural scaffold, for example. The porous drug-eluting coating 310 may be manufactured of such material as to prevent tissue in-growth, which may potentially interfere with the lead/electrode function. The porous drug-eluting coating 310 may be formed such that a porous scaffold coating is created. The porous drug-eluting coating 310 may be sufficiently conductive so as to allow sufficient electrical sensing or shock to penetrate the coating.

In addition to the porous polytetrafluoroethylene (PTFE), the porous drug-eluting coating 310 comprises a biodegradable polymer and one or more therapeutic agents, or drugs. In one embodiment, the porous drug-eluting coating 310 components are admixed, for example, with a solvent to provide a solution or mixture. In one embodiment, the solvent does not interfere with the activity of the drug. Examples of such solvents include water, alcohol, cyclohexanone, acetone and combinations thereof. The solution can be applied to at least a portion or all of a lead 104 and/or one or more electrodes 208 and 210, for example, by spray coating. After the solvent in the solution is evaporated, a drug-eluting polymer comprising at least one drug and a biodegradable polymer, remains within the pores of the porous drug-eluting coating 310. The process can be repeated as many times as desired. Alternatively, the porous drug-eluting coating 310 can be disposed by dip-coating. Brush-coating can also be used. RF magnetron physical vapor deposition sputtering process may also be employed. The porous drug-eluting coating 310 may also be applied using a combination of spraying, dipping, sputtering and/or brushing, for example. Further, the coating 310 may be disposed by injecting with a syringe in-situ, for example.

In one embodiment, the porous drug-eluting coating 310 comprises one or more layers ranging from about submicron to about 10 microns in thickness, about 1 to about 50 microns in thickness or about 50 to about 100 microns in thickness. In another embodiment, the thickness of the coating 310 ranges from about 1 to about 5 microns, about 5 to about 10 microns, about 10 to about 15 microns, about 15 to about 20 microns, about 20 to about 30 microns, about 30 to about 40 microns, about 40 to about 50 microns, about 50 to about 60 microns, about 60 to about 70 microns, about 70 to about 80 microns, about 80 to about 90 microns, or about 90 to about 100 microns.

The biodegradable polymer utilized in the porous drug-eluting coating 310 may be comprised of polylactic acid and its derivatives, polyglycolic acid and its derivatives, polycaprolactum, copolymers of lactic acid, glycolic acid and caprolactum, polyethylene glycol, hyaluranic acid and its derivatives, phoshorylcholine, polyvinylpyrrolidone (PVP) and combinations thereof. The degradation of such polymers or combination of polymers can be controlled by proper selection and allows for optimization of the drug efficacy and therapeutic effect.

The therapeutic agent or drug utilized in the porous drug-eluting coating 310 include, but is not limited to an anti-inflammatory, anti-proliferative, anti-arrhythmic, anti-migratory, anti-neoplastic, antibiotic, anti-restenotic, anti-coagulation, anti-clotting (e.g., heparin, coumadin, aspirin), anti-thrombogenic or immunosuppressive agent, or an agent that promotes healing, such as a steroid (e.g., a glucocorticosteriod), and/or re-endothelialization or combinations thereof.

Any drug or bioactive agent which can serve a useful therapeutic, prophylactic or even diagnostic function when released into a patient can be used. The agents may be used alone, in combinations of agents, admixed or chemically bound with the coating 310.

More specifically, the therapeutic agents may include, but are not limited to paclitaxel, clobetasol, rapamycin (sirolimus), everolimus, tacrolimus, actinomycin-D, dexamethasone (e.g., dexamethasone sodium phosphate or dexamethasone sodium acetate), mometasone furoate, hyaluronic acid, vitamin E, mycophenolic acid, cyclosporins, beclomethasone (e.g., beclomethasone dipropionate anhydrous), their derivatives, analogs, salts or combinations thereof.

In one embodiment, a combination of an anti-proliferative (e.g., everolimus or paclitaxel) and an anti-inflammatory (e.g., dexamethasone, clobetasol or mometasone furoate) agent may be employed. In one embodiment, a combination of dexamethasone and everolimus is employed. In another embodiment, a combination of clobetasol and everolimus is employed. In yet another embodiment, a combination of dexamethasone and paclitaxel is employed. In another embodiment, a combination of clobetasol and paclitaxel is employed. In another embodiment, a combination of dexamethasone and sirolimus is employed. In one embodiment a combination of clobetasol and sirolimus is employed.

Additional suitable agents can be found in the Physicians Desk Reference (PDR) (see, for example, The Physicians Desk Reference (59th ed. 2005).

The therapeutic agent can be present in any effective amount. An “effective amount” generally means an amount which provides the desired local or systemic effect. For example, an effective dose is an amount sufficient to affect a beneficial or desired clinical result. The precise determination of what would be considered an effective dose may be based on factors individual to each patient, including their size and age. In one embodiment, the therapeutic agent is present in a concentration of less than about 100 μg/cm². For example, the agent may be present in a range of about 2 to about 10 μg/cm², about 10 to about 20 μg/cm², about 20 to about 30 μg/cm², about 30 to about 40 μg/cm², about 40 to about 50 μg/cm², about 50 to about 60 μg/cm², about 60 to about 70 μg/cm², about 70 to about 80 μg/cm², about 80 to about 90 μg/cm² and/or about 90 to about 100 μg/cm². The agent(s) may also be present at a concentration of higher than about 100 μg/cm².

In an embodiment, the therapeutic agent is available immediately after and/or during implantation (time of injury). In another embodiment, within a few days, such as about 1 to about 5 days, following implantation, the agent has nearly completely eluted. In another embodiment, the therapeutic agent elutes in a couple of hours to several days to several weeks (e.g., in about 1 to about 5 weeks). The therapeutic agent may also be designed to have longer eluting times, such as several months. Additionally, the lead may be designed so that one therapeutic agent is released at the time of implantation (time of injury), while another therapeutic agent releases more slowly, for example, over the course of about several weeks to about a month or two from the time of implantation. In one embodiment, the therapeutic agents may be the same or different therapeutic agents.

The porous drug-eluting coating embodiments may provide a controllable drug-eluting means for therapeutic response to tissue damage due to implantation and shocking of medical leads. Further, the coatings provided encourage a positive biological reaction to the electrical stimulation and its effects on surrounding tissue.

It is to be understood that the above description is intended to be illustrative, and not restrictive. It should be noted that the above text discusses and figure illustrate, among other things, implantable leads for use in cardiac situations; however, the present leads and methods are not so limited. Many other embodiments and contexts, such as for non-cardiac nerve and muscle situations or for external nerve and muscle situations, will be apparent to those of skill in the art upon reviewing the above description. The scope should, therefore, be determined with reference to the appended claims, along with the full scope of legal equivalents to which such claims are entitled.

What is claimed is:

1. A tachy lead comprising:
a lead body extending from a lead proximal end portion to a lead distal end portion, and having an intermediate portion therebetween;
one or more tissue sensing/stimulation electrodes disposed along the lead body;
one or more terminal connections disposed along the lead proximal end portion;
one or more conductors contained within the lead body extending between the tissue sensing/stimulation electrodes and the terminal connections;
a porous drug-eluting coating disposed onto at least a portion of the lead body and/or sensing/stimulation electrodes; and
wherein the porous drug-eluting coating comprises:
porous polytetrafluoroethylene (PTFE);
a biodegradable polymer; and
one or more drugs.

2. The tachy lead of claim 1, wherein at least one of the one or more tissue sensing/stimulation electrodes is a defibrillation shocking coil electrode.

3. The tachy lead of claim 1, wherein the one or more tissue sensing/stimulation electrodes is selected from the group of anode, cathode, defibrillation shocking coil electrode, or combinations thereof.

4. The tachy lead of claim 1, wherein the rate of drug-elution from the porous drug-eluting coating may be controlled.

5. The tachy lead of claim 1, wherein the biodegradable polymer comprises polylactic acid and its derivatives, polyglycolic acid and its derivatives, polycaprolactum, copolymers of lactic acid, glycolic acid and caprolactum, polyethylene glycol, hyaluranic acid and its derivatives, phoshorylcholine, polyvinylpyrrolidone (PVP) or combinations thereof.

6. The tachy lead of claim 1, wherein the one or more drugs comprise an anti-inflammatory, anti-proliferative, anti-arrhythmic, anti-migratory, anti-neoplastic, antibiotic, anti-restenotic, anti-coagulation, anti-clotting, anti-thrombogenic or immunosuppressive agent, or an agent that promotes healing and/or re-endothelialization or combinations thereof.

7. The tachy lead of claim 1, wherein the one or more drugs comprise paclitaxel, clobetasol, rapamycin (sirolimus), everolimus, tacrolimus, actinomycin-D, dexamethasone, mometasone furoate, hyaluronic acid, vitamin E, mycophenolic acid, cyclosporins, beclomethasone, their derivatives, analogs, salts or combinations thereof.

8. A tachy lead comprising:
a lead body extending from a lead proximal end portion to a lead distal end portion, and having an intermediate portion therebetween;
one or more tissue sensing/stimulation electrodes disposed along the lead body;
one or more defibrillation shocking coil electrodes;
one or more terminal connections disposed along the lead proximal end portion;
one or more conductors contained within the lead body extending between the tissue sensing/stimulation electrodes and the terminal connections;
a porous drug-eluting coating disposed onto at least a portion of the lead body and/or the one or more defibrillation shocking coil electrodes; and
wherein the porous drug-eluting coating comprises:
porous polytetrafluoroethylene (PTFE);
a biodegradable polymer; and
one or more drugs.

9. A lead system comprising:
one or more tachy leads, each tachy lead comprising:
a lead body;
one or more electrodes disposed along the lead body;
a porous drug-eluting coating disposed onto at least a portion of the lead body and/or electrodes;
wherein the porous drug-eluting coating comprises:
porous polytetrafluoroethylene (PTFE);
a biodegradable polymer; and
one or more drugs; and
an implantable medical device, electrically coupled to the one or more tachy leads.

10. The lead system of claim 9, further comprising an energy source coupled to the implantable medical device.

11. The lead system of claim 9, wherein the rate of drug-elution from the porous drug-eluting coating may be controlled.

12. The lead system of claim 9, wherein the at least one of the one or more electrodes is a defibrillation shocking coil electrode.

13. The lead system of claim 9, wherein the biodegradable polymer comprises polylactic acid and its derivatives, polyglycolic acid and its derivatives, polycaprolactum, copolymers of lactic acid, glycolic acid and caprolactum, polyethylene glycol, hyaluranic acid and its derivatives, phoshorylcholine, polyvinylpyrrolidone (PVP) or combinations thereof.

14. The lead system of claim 9, wherein the one or more drugs comprise an anti-inflammatory, anti-proliferative, anti-arrhythmic, anti-migratory, anti-neoplastic, antibiotic, anti-restenotic, anti-coagulation, anti-clotting, anti-thrombogenic or immunosuppressive agent, or an agent that promotes healing and/or re-endothelialization or combinations thereof.

15. The lead system of claim 9, wherein the one or more drugs comprise paclitaxel, clobetasol, rapamycin (sirolimus), everolimus, tacrolimus, actinomycin-D, dexamethasone, mometasone furoate, hyaluronic acid, vitamin E, mycophenolic acid, cyclosporins, beclomethasone, their derivatives, analogs, salts or combinations thereof.

16. A method of manufacturing a lead, the method comprising:
forming a tachy lead; and
disposing a porous drug-eluting coating on all or a portion of the lead;
wherein the drug-eluting coating comprises:
porous polytetrafluoroethylene (PTFE)
a biodegradable polymer; and
one or more drugs.

17. The method of claim 16, wherein disposing the porous drug-eluting coating on all or a portion of the lead includes disposing on all or a portion of one or more electrodes.

18. The method of claim 17, wherein at least one of the one or more electrodes is a defibrillation shocking coil electrode.

19. The method of claim 16, wherein disposing a porous drug-eluting coating includes spraying, dipping, sputtering or brushing.

20. The method of claim 16, wherein disposing a porous drug-eluting coating includes injecting with a syringe in-situ.

21. The method of claim 16, wherein the biodegradable polymer comprises polylactic acid and its derivatives, polyglycolic acid and its derivatives, polycaprolactum, copolymers of lactic acid, glycolic acid and caprolactum, polyethylene glycol, hyaluranic acid and its derivatives, phoshorylcholine, polyvinylpyrrolidone (PVP) or combinations thereof.

22. The method of claim 16, wherein the one or more drugs comprise an anti-inflammatory, anti-proliferative, anti-arrhythmic, anti-migratory, anti-neoplastic, antibiotic, anti-restenotic, anti-coagulation, anti-clotting, anti-thrombogenic or immunosuppressive agent, or an agent that promotes healing and/or re-endothelialization or combinations thereof.

23. The method of claim 16, wherein the one or more drugs comprise paclitaxel, clobetasol, rapamycin (sirolimus), everolimus, tacrolimus, actinomycin-D, dexamethasone, mometasone furoate, hyaluronic acid, vitamin E, mycophenolic acid, cyclosporins, beclomethasone, their derivatives, analogs, salts or combinations thereof.
